(19) United States
(12) Patent Application Publication
(10) Pub. No.: US 2026/0283931 A1
(43) Pub. Date: Sep. 24, 2026

(54) COMPOSITION BASED ON CHITOSAN AND A SPECIFIC COMPOUND

(71) Applicant: L'OREAL, Paris (FR)

(72) Inventors: Danielle LE VERGE, Chevilly Larue (FR); Bernadette DAVID, Chevilly Larue (FR); Christophe KUSINA, Chevilly Larue (FR)

(73) Assignee: L'OREAL, Paris (FR)

(21) Appl. No.: 19/474,000

(22) PCT Filed: Apr. 18, 2024
(86) PCT No.: PCT/EP2024/060545

§ 371 Date: Oct. 9, 2025

(30) Foreign Application Priority Data
Apr. 18, 2023 (EP) 23305594.6

(51) Int. Cl. A61K 8/73; A61K 8/36; A61K 8/44; A61K 8/46; A61Q 1/02; A61Q 5/06; A61Q 19/00

(52) U.S. Cl. CPC A61K 8/736; A61K 8/361; A61K 8/442; A61K 8/466; A61Q 1/02; A61Q 5/06; A61Q 19/00; A61K 2800/34; A61K 2800/43

(57) ABSTRACT

The present invention relates to a cosmetic composition comprising, in a physiologically acceptable aqueous medium:
a) at least 0.01% by weight of native chitosan, with respect to the total weight of the composition, which native chitosan has a molecular weight strictly greater than 3000 Daltons, this amount being strictly less than 15% by weight; and
b) at least one compound chosen from among anionic surfactants of the carboxylate, phosphate, sulfonate, or sulfate type and/or mixtures thereof.

The present invention relates to a cosmetic composition comprising, in a physiologically acceptable aqueous medium:
a) at least 0.01% by weight of native chitosan, with respect to the total weight of the composition, which native chitosan has a molecular weight strictly greater than 3000 Daltons, this amount being strictly less than 15% by weight, and
b) at least one compound chosen from among anionic surfactants of the carboxylate, phosphate, sulfonate, or sulfate type and/or mixtures thereof.

The invention further relates to a method for making up and/or caring for the skin and/or keratinous appendages, wherein the composition according to the invention is applied to the skin and/or keratinous appendages.

There are many cosmetic compositions for which resistance properties of the deposited film, after application to keratinous matter, are desired. Examples include lipsticks or nail varnishes. In order to obtain such a result, particular raw materials, in particular film-forming agents, can be combined. However, the presence of such agents for increasing the resistance of the film on keratinous matter can lead to compositions that cause dryness. Moreover, the film-forming agents commonly used are typically petrochemical in nature, such as silicones or acrylic polymers.

In other respects, the aim is often to obtain covering compositions.

Chitosan-based systems show that the combination of chitosan and a pigment can be used to generate films that are resistant to both dry and oily friction. Nevertheless, the sensitivity of these deposits to water is very high, and remains a major challenge for chitosan-based technologies.

The formulator is therefore looking for raw materials and/or systems enabling fluid (i.e. that can flow) chitosan-based compositions to be obtained, the deposit whereof is covering, homogeneous, durable and more resistant to water.

Moreover, the formulation of environmentally-friendly cosmetic products, i.e. those whose design and development take account of environmental issues, is becoming a major concern in helping to meet global challenges.

It has therefore become essential to propose more sustainable compositions and/or preparation processes and/or ingredients to meet these environmental challenges.

In this context, it is important to develop novel cosmetic compositions with a better carbon footprint, in particular by promoting the use of renewable raw materials and/or those with a good wilderness quality index and/or of natural origin, and more particularly those of plant origin, while reducing the use of compounds of petrochemical origin.

The purpose of the present invention is to propose aqueous cosmetic compositions, whether colored or not, with good properties, particularly in terms of homogeneity and durability, especially with good durability/resistance to water.

After application, these compositions leave a film-forming deposit with good coverage and homogeneity, and good resistance to wear. The colored films formed are adhesive and cohesive, and have improved water resistance.

These compositions further include sustainable ingredients, helping to meet environmental challenges.

The present invention thus relates to a cosmetic composition, in particular for making-up and/or caring for the skin and/or lips, in particular the skin, comprising, in a physiologically acceptable aqueous medium:
a) at least 0.01% by weight of native chitosan, with respect to the total weight of the composition, which native chitosan has a molecular weight strictly greater than 3000 Daltons, this amount being strictly less than 15% by weight, and
b) at least one compound chosen from among anionic surfactants of the carboxylate, phosphate, sulfonate, or sulfate type and/or mixtures thereof.

“Physiologically acceptable” refers to a medium compatible with keratin matter.

It further relates to a method for making up and/or caring for the skin and/or keratinous appendages, wherein the composition according to the invention is applied to the skin and/or keratinous appendages.

Preferably, the composition according to the invention is substantially free of (meth)acrylate polymer. Preferably, the composition according to the invention is substantially free of silicone.

“Substantially free of (meth)acrylate polymer” means that the composition comprises less than 1% by weight, relative to the total weight of the composition, preferably less than 0.5% by weight, preferably less than 0.3% by weight, and more preferably 0.1% by weight of (meth)acrylate polymer. Preferably, the composition is totally free of (meth)acrylate polymer. “(Meth)acrylate polymer” means a polymer comprising at least an acrylic acid monomer or at least a methacrylic acid monomer.

“Substantially free of silicone” means that the composition comprises less than 1% by weight, relative to the total weight of the composition, preferably less than 0.5% by weight, preferably less than 0.3% by weight, and more preferably 0.1% by weight of silicone. Preferably, the composition is totally free of silicone. “Silicone” means any silicone compound.

### Chitosan

The composition according to the invention comprises at least 0.01% by weight, relative to the total weight of the composition, of at least one native chitosan having a molecular weight strictly greater than 3000 Daltons (3 kDa).

The amount of native chitosan is strictly less than 15% by weight relative to the total weight of the composition.

Preferably, the native chitosan has a molecular weight greater than or equal to 10 kDa, preferably greater than or equal to 15 kDa, and preferably greater than or equal to 20 kDa.

Preferably, the native chitosan has a molecular weight between 10 kDa and 2 MDa, preferably between 15 kDa and 1.5 MDa, preferably between 20 kDa and 300 kDa, and preferably between 20 kDa and 200 kDa.

Chitosan is not widely found in nature. It has only been reported in the exoskeletons of certain insects, such as termite queens, and in the cell walls of a particular class of fungi, namely zygomycetes.

Chitosan is obtained by deacetylation of chitin. Chitin is a polysaccharide composed of several N-acetyl-D-glucosamine units interlinked by a β-(1,4) type bond.

The ideal chemical structure of chitosan is a chain of β-D-glucosamine monomers linked by a glycoside (1->4) bond.

According to the invention, “chitosan” means any copolymer made up of N-acetyl-D-glucosamine and D-glucosamine constituent units, the degree of acetylation whereof is less than 90%. Chitosan consists of glucosamine sugar units (deacetylated units) and N-acetyl-D-glucosamine units (acetylated units) interlinked by β (1,4) type bonds and represents a polymer of the poly(N-acetyl-D-glucosamine)-poly(D-glucosamine) type.

Preferably, the degree of acetylation of chitosan is less than or equal to 80%, preferably less than or equal to 70%, preferably less than or equal to 60%, preferably less than or equal to 50%, preferably less than or equal to 35%, preferably less than or equal to 25%, and preferably less than or equal to 15%.

The degree of acetylation is the percentage of acetylated units relative to the total number of units, and can be determined by Fourier transform infrared spectroscopy (IFTIR) or titration with a strong base.

The chitosan of the invention is preferably a polysaccharide prepared from a fungal source. It is in particular extracted and purified from safe and abundant food-grade or biotechnological fungal sources such as Agaricus Bisporus or Aspergillus Niger.

The chitosan of the invention is preferably derived from the mycelium of a fungus of the Ascomycete type, in particular Aspergillus niger and/or a Basidiomycete fungus, in particular Lentinula edodes (shiitake) and/or Agaricus bisporus. Preferably the fungus is Aspergillus niger.

The chitosan can be of GMO origin, but is preferably of non-GMO origin.

The chitosan according to the invention is native, i.e. it is not modified. In particular, it has not undergone any chemical modification.

One method for preparing chitosan is described in the international patent application WO03068824.

Preferably, the chitosan used in the invention is in powder form. It is marketed by Kitozyme under the name Kiosmetine or Kionutrime.

The chitosan is preferably present in an amount ranging from 0.01% to 14% by weight, preferably from 0.1% to 14% by weight, preferably from 0.1% to 12% by weight, preferably from 0.2% to 7% by weight, and preferably from 0.25% to 5% by weight relative to the total weight of the composition.

### Anionic Surfactants

The composition according to the present invention comprises one or more anionic surfactants.

The term anionic surfactant means a surfactant that comprises in terms of ionic or ionizable groups only anionic groups.

In this description, an entity is qualified as being “anionic” when it has at least one permanent negative charge or when it can be ionized as a negatively charged entity, in the conditions of use of the composition of the invention (medium, pH for example) and not comprising any cationic charge.

The one or more anionic surfactants used in the invention are chosen from sulfate, sulfonate, carboxylic (or carboxylate), and phosphate surfactants, and from mixtures thereof.

According to the invention, the weight ratio of chitosan to anionic surfactant (i.e. the weight ratio chitosan:anionic surfactant) is between 0.1 and 10. Preferably, it is between 0.2 and 10, more preferably between 0.3 and 5, and even more preferably between 0.5 and 3.

### Carboxylate Surfactant

The composition according to the invention can comprise a carboxylate anionic surfactant. The carboxylate anionic surfactants that can be used in the composition of the invention include at least one carboxylic or carboxylate function (—COOH or —COO—).

Preferably, the carboxylate anionic surfactants are chosen from citrates, fatty acids, N-acyl amino acids, lactylates and their salts.

The one or more carboxylate anionic surfactants can be present in the composition in a content ranging from 0.1% to 10%, preferably from 0.1% to 5%, more preferably from 0.1 to 3%, even more preferably from 0.45 to 2%, and even better from 0.75% to 1.5% by weight relative to the total weight of the composition.

Preferably, the composition according to the invention comprises at least one carboxylate anionic surfactant chosen from fatty acids and/or their salts.

“Fatty acid” means a carboxylic acid with an aliphatic chain comprising 8 to 30 carbon atoms, preferably 12 to 24 carbon atoms, preferably 12 to 18 carbon atoms and more preferably 16 to 18 carbon atoms. The fatty acid can be liquid or non-liquid.

A liquid fatty acid means a fatty acid that is liquid at room temperature (25° C.) and at atmospheric pressure (760 mm Hg, i.e. 1,013.10⁵ Pa).

The fatty acids of the invention can be saturated or unsaturated.

Liquid saturated fatty acids can optionally be branched. They can optionally include at least one aromatic ring in their structure. They are preferably acyclic. Isostearic acid is one particular example.

Unsaturated fatty acids have at least one double or triple bond in their structure, and preferably one or more double bonds. When multiple double bonds are present, there are preferably 2 or 3, and they may or may not be conjugated. These unsaturated fatty acids can be linear or branched. They can optionally include at least one aromatic ring in their structure. They are preferably acyclic.

Fatty acids suitable for implementing the invention are more particularly chosen from linear or branched, saturated or unsaturated acids containing from 8 to 30 carbon atoms.

Preferably, the fatty acid is chosen from stearic acid, palmitic acid, palmitoleic acid, linoleic acid, caprylic acid, myristic acid, lauric acid, behenic acid and isostearic acid.

Examples of fatty acids include palmitic acid marketed by Cayman Chemical, palmitic acid available under the trade name EDENOR C16-98 MY, oleic acid available under the trade name EDENOR OL 75 MY, isostearic acid available under the trade name PRISORINE 3505-LQ-(GD), and behenic acid available under the trade name PALMERA A8522.

The fatty acid can be in a free acid form or in a partially or fully salified form. When it is in salified form, this involves alkali or amine salts such as sodium, potassium or lithium salts; alkaline earth salts such as calcium, magnesium or strontium salts; aluminum, zirconium or zinc salt; ammonium salt, amino alcohol salt such as (2-hydroxy ethyl) salt, diethanolamine salt, triethanolamine salt or triisopropanolamine salt. Preferably, the salts are aluminum, magnesium, calcium, zirconium, zinc, sodium or potassium salts.

Examples include sodium or potassium caprate, sodium or potassium caprylate, sodium or potassium or magnesium or calcium or ammonium or triethanolamine laurate, sodium or potassium or magnesium or calcium or diethanolamine or triethanolamine or triisopropanolamine myristate, sodium or potassium or magnesium or triethanolamine palmitate, sodium or potassium or magnesium or triethanolamine cocoate, sodium or potassium or magnesium or calcium or ammonium or diethanolamine or triethanolamine stearate, sodium or potassium or ammonium oleate, sodium arachidate or sodium or potassium or calcium behenate.

According to another embodiment, the composition according to the invention comprises a carboxylate anionic surfactant chosen from N-acyl amino acids and their salts.

N-acyl amino acid is an amino acid with an acyl group on the nitrogen of the amino acid.

The amino acid can be, for example, lysine, glycine, glutamic acid or alanine. Preferably, the amino acid is selected from glycine or glutamic acid, more preferably the amino acid is glutamic acid.

The N-acyl amino acid can be in free acid form or in partially or fully salified form. When it is in salified form, this involves alkali salts such as sodium, potassium or lithium salts; alkaline earth salts such as calcium, magnesium or strontium salts; aluminum, zirconium or zinc salt; ammonium salt or amino alcohol salt such as (2-hydroxy ethyl) ammonium salt. Preferably, the salts are aluminum, magnesium, calcium, zirconium, zinc, sodium or potassium salts.

The N-acyl amino acid can comprise an acyl group having from 8 to 22 carbon atoms, such as, for example, a 2-ethylhexanoyl, caproyl, lauroyl, myristoyl, palmitoyl, stearoyl or cocoyl group. Preferably, the acyl group is a stearoyl group.

One example is sodium stearoyl glutamate, marketed under the trade name AMISOFT HS 11PF or disodium stearoyl glutamate marketed under the trade name AMISOFT HS 21P.

Preferably, the carboxylate anionic surfactant is a glutamic acid derivative and/or a salt thereof, and more particularly a stearoyl glutamate, for example disodium stearoyl glutamate, sodium stearoyl glutamate or aluminum stearoyl glutamate.

Preferably, the anionic surfactant is disodium stearoyl glutamate.

According to another particular embodiment, the composition according to the invention comprises a carboxylate anionic surfactant that is a derivative of glycine and/or a salt thereof.

Preferably, said glycinate anionic surfactant is a C6-C24 acylglycinate, in particular a C10-C20 acylglycinate, more preferably a cocoylglycinate, even more preferably an alkali metal salt of cocoylglycinate, and even more preferably sodium cocoyl glycinate.

For example, sodium cocoyl glycinate is marketed under the trade name Amilite® GCS by Ajinomoto.

According to another embodiment, the carboxylate anionic surfactant is chosen from among citrates.

Preferably, the carboxylate anionic surfactant is glyceryl stearate citrate available under the trade name AXOL C 62 PELLETS.

The one or more fatty acids, the N-acyl amino acids, the citrates and/or the salts thereof can be present in the composition in a content ranging from 0.1% to 10%, preferably from 0.1% to 5%, more preferably from 0.1 to 3%, even more preferably from 0.45 to 2%, and even better from 0.75% to 1.5% by weight relative to the total weight of the composition.

According to the invention, the weight ratio of chitosan to carboxylate anionic surfactant (i.e. the weight ratio chitosan:anionic surfactant) is between 1 and 10. Preferably, it is between 1 and 5, more preferably between 1 and 3, and even more preferably between 1 and 2.

### Sulfonate Surfactant

The composition according to the invention can comprise a sulfonate anionic surfactant.

The sulfonate anionic surfactants that can be used in the composition of the invention include at least one sulfonate function (—SO3H or —SO3⁻).

They can be chosen from the following compounds: alkylsulfonates, alkylamidesulfonates, alkylarylsulfonates, alpha-olefin-sulfonates, paraffin-sulfonates, alkylsulfosuccinates, alkylethersulfosuccinates, alkylamidesulfosuccinates, alkylsulfoacetates, N-acyltaurates, acylisethionates; alkylsulfolaurates; as well as the salts of these compounds; the alkyl groups of these compounds comprising from 6 to 30 carbon atoms, in particular from 12 to 28, even better from 14 to 24, or even from 16 to 22 carbon atoms; the aryl group preferably designating a phenyl or benzyl group; these compounds able to be polyoxyalkylenated, in particular polyoxyethylenated and then comprising preferably from 1 to 50 ethylene oxide units, better from 2 to 10 ethylene oxide units.

Preferably, the surfactant is a sulfosuccinate anionic surfactant chosen from alkyl sulfosuccinates, alkyl ether sulfosuccinates, and alkyl amide sulfosuccinates; more preferably it is an alkyl sulfosuccinate.

Preferably, said sulfosuccinate anionic surfactant is a C6-C24 alkylsulfosuccinate, more preferably a laurylsulfosuccinate, even more preferably an alkali metal salt of laurylsulfosuccinate, and even more preferably disodium lauryl sulfosuccinate.

For example, disodium lauryl sulfosuccinate is marketed by Evonik under the trade name Rewopol® SB 12 P.

A composition according to the invention further comprises an isethionate anionic surfactant.

Preferably, said isethionate anionic surfactant is a C6-C24 acylisethionate, in particular a C10-C20 acylisethionate, more preferably a cocoyl isethionate, even more preferably an alkali metal salt of cocoyl isethionate, and even more preferably sodium cocoyl isethionate. For example, sodium cocoyl isethionate is marketed by Clariant under the trade name Hostapon® SCI or by BASF under the trade name Jordapon® CI.

When the one or more sulfonate anionic surfactants are in salt form, said salt can be chosen from alkali metal salts such as sodium or potassium salt, ammonium salts, amine salts and in particular aminoalcohols, alkaline earth metal salts such as magnesium salt, and mixtures thereof.

The one or more sulfonate anionic surfactants can be present in the composition in a content ranging from 0.1% to 10%, preferably from 0.1% to 5%, more preferably from 0.1 to 3%, even more preferably from 0.45 to 2%, and even better from 0.75% to 1.5% by weight relative to the total weight of the composition.

According to the invention, the weight ratio of chitosan to sulfonate anionic surfactant (i.e. the weight ratio chitosan:anionic surfactant) is between 1 and 10. Preferably, it is between 1 and 5, more preferably between 1 and 3, and even more preferably between 1 and 2.

### Sulfate Surfactant

The composition according to the invention can comprise a sulfate anionic surfactant.

The sulfate anionic surfactants that can be used in the composition of the invention include one or more sulfate functions (—OSO3H or —OSO3⁻).

Such surfactants can advantageously be chosen from alkylsulfates, alkylethersulfates, alkylamidosulfates, alkylamidoethersulfates, alkylarylpolyethersulfates, and monoglyceride sulfates, as well as their salts and mixtures thereof; the alkyl groups of these compounds comprise in particular from 6 to 30 carbon atoms, preferably from 8 to 26, and more preferably from 10 to 22 carbon atoms.

Preferably, the one or more sulfate anionic surfactants are chosen from alkylsulfates, in particular C8 to C26 alkylsulfates, and preferably from C10 to C22 alkylsulfates.

Examples of sulfate anionic surfactants include sodium coco sulfate, sodium lauryl sulfate and sodium laureth sulfate.

Preferably, the compound chosen from the anionic surfactants is sodium coco sulfate marketed under the trade name RHODAPON CAS 100N MB.

When the one or more sulfate anionic surfactants are in salt form, said salt can be chosen from alkali metal salts such as sodium or potassium salt, ammonium salts, amine salts and in particular aminoalcohols, alkaline earth metal salts such as magnesium salt, and mixtures thereof.

The one or more sulfate anionic surfactants can be present in the composition in a content ranging from 0.1% to 10%, preferably from 0.1% to 5%, more preferably from 0.1 to 3%, even more preferably from 0.45 to 2%, and even better from 0.75% to 1.5% by weight relative to the total weight of the composition.

According to the invention, the weight ratio of chitosan to sulfate anionic surfactant (i.e. the weight ratio chitosan:anionic surfactant) is between 1 and 10. Preferably, it is between 1 and 5, more preferably between 1 and 3, and even more preferably between 1 and 2.

### Pigmentary Coloring Matter

The composition according to the invention can further comprise at least one pigmentary coloring matter. This pigmentary coloring matter is chosen from pulverulent coloring matter, such as mineral pigments, nacres and organic pigments.

The term “pigments” should be understood to mean white or colored, mineral or organic particles, which are insoluble in an aqueous solution and are intended for coloring the composition and/or the resulting deposit. Preferably the pigment is a colored (i.e. not white) mineral or organic particle insoluble in an aqueous solution and intended for coloring the composition and/or the resulting deposit.

The coloring matter can be present in the composition in a content ranging from 1% to 70% by weight, relative to the total weight of the composition, preferably from 1% to 60%, preferably from 3% to 50%, preferably from 3% to 45% by weight, relative to the weight of the composition, preferably from 4% to 30% by weight, preferably from 5% to 20% by weight, and preferably from 6% to 15% by weight.

### Mineral Pigments

According to one particular embodiment, the pigments used according to the invention are selected from mineral pigments.

“Mineral pigment” means any pigment that complies with the definition in Ullmann's Encyclopedia in the Inorganic Pigment chapter. Mention can be made, among the mineral pigments useful in the present invention, of zirconium or cerium oxides, along with zinc, iron (black, yellow or red) or chromium oxides, manganese violet, ultramarine blue, chromium hydrate and iron blue, titanium dioxide, metallic powders such as aluminum powder and copper powder. The following mineral pigments can also be used: Ta2O5, Ti3O5, Ti2O3, TiO, ZrO2 in a mixture with TiO2, ZrO2, Nb2O5, CeO2, ZnS.

The size of the pigment useful in the context of the present invention is in general greater than 100 nm and may range up to 10 μm, preferably from 200 nm to 5 μm, and more preferably from 300 nm to 1 μm. According to a particular embodiment of the invention, the pigments have a size characterized by a D[50] greater than 100 nm and may range up to 10 μm, preferably from 200 nm to 5 μm, and more preferably from 300 nm to 1 μm. The sizes are measured by measured by static diffusion of light by means of a commercial MasterSizer 3000® granulometer from Malvern, making it possible to apprehend the granulometric distribution of all the particles over a wide range that can range from 0.01 μm to 1000 μm. The data are processed on the basis of the classic Mie scattering theory. This theory is the most adapted for size distributions ranging from submicron to multi-micron, and makes it possible to determine an “effective” diameter of particles. This theory is described particularly in the book by Van de Hulst, H. C., “Light Scattering by Small Particles”, Chapters 9 and 10, Wiley, New York, 1957. D[50] represents the maximum size of 50% by volume of the particles.

In the context of the present invention, the mineral pigments are more particularly iron oxide and/or titanium dioxide.

As mineral pigments that can be used in the invention, nacres can also be cited.

The term “nacres” should be understood to mean iridescent or non-iridescent colored particles of any shape, which are in particular produced by certain mollusks in their shell or else are synthesized and which exhibit a color effect by optical interference.

The nacres may be selected from pearlescent pigments such as titanium mica coated with iron oxide, titanium mica coated with bismuth oxychloride, titanium mica coated with chromium oxide, titanium mica coated with an organic dye, and pearlescent pigments based on bismuth oxychloride. This may also involve mica particles at the surface whereof are superposed at least two successive layers of metal oxides and/or of organic dyes. By way of example of nacres, mention may also be made of natural mica coated with titanium oxide, with iron oxide, with natural pigment or with bismuth oxychloride. The nacres may more particularly possess a yellow, pink, red, bronze, orange, brown and/or copper color or glint.

Among the pigments that can be used according to the invention, mention can also be made of those that have an optical effect that differs from a simple conventional hue effect, i.e. a unified and stabilized effect of the kind produced by conventional dyes, such as, for example, monochromatic pigments.

For the purpose of the invention, “stabilized” means absence of an effect of variability of color with the angle of observation or in response to a temperature change. For example, this material may be selected from particles having a metallic glint, goniochromatic coloring agents, diffracting pigments, thermochromatic agents, optical brighteners, and also fibers, in particular of the interference type. Of course, these various materials may be combined so as to provide the simultaneous manifestation of two effects, or even a new effect in accordance with the invention.

According to one particular embodiment, the composition according to the invention comprises at least one non-coated pigment.

### Organic Pigments

According to another embodiment of the invention, the pigmentary coloring matter is an organic, synthetic, natural pigment or pigment of natural origin.

“Organic pigment” means any pigment that complies with the definition in Ullmann's Encyclopedia in the Organic Pigment chapter. The organic pigments can in particular be selected from the compounds nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, quinophthalone.

The organic pigment or pigments can be selected for example from carmine, carbon black, aniline black, melanin, azo yellow, quinacridone, phthalocyanine blue, sorghum red, the blue pigments codified in the Color Index under the references CI 42090, 69800, 69825, 73000, 74100, 74160, the yellow pigments codified in the Color Index under the references CI 11680, 11710, 15985, 19140, 20040, 21100, 21108, 47000, 47005, the green pigments codified in the Color Index under the references CI 61565, 61570, 74260, the orange pigments codified the Color Index under the references CI 11725, 15510, 45370, 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, 75470, and the pigments obtained by oxidative polymerization of indole or phenolic derivatives as described in patent FR 2 679 771.

The pigments can also be in the form of composite pigments as described in patent EP 1 184 426. These composite pigments may be composed in particular of particles including an inorganic core covered at least partially with an organic pigment and at least one binder for fixing the organic pigments to the core.

The pigment may also be a lacquer.

The term lacquer means insoluble dyes adsorbed on insoluble particles, the assembly thus obtained remaining insoluble during use.

The inorganic substrates onto which the dyes are adsorbed are for example alumina, silica, borosilicate, calcium and sodium or calcium aluminum borosilicate, and aluminum.

Among the organic dyes, mention can be made of cochineal carmine. The products known under the following names can also be cited: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), D&C Blue 1 (CI 42 090). By way of examples of lacquers, the product known by the name D&C Red 7 (CI 15 850:1) can be cited.

### Fillers

The composition according to the invention can further comprise at least one filler.

The term “fillers” should be understood to denote inorganic or synthetic, colorless or white particles of any shape, insoluble and dispersed in the medium of the composition regardless of the temperature at which the composition is manufactured. Generally speaking, the fillers included in the compositions according to the invention are not pigmentary coloring matters.

Preferably, the filler is chosen from cellulose particles, silicas, starch, kaolin, clays, nylon or polymethyl methacrylate (PMMA) particles and mixtures thereof.

The cellulose particles that can be used according to the invention are preferably spherical (cellulose beads).

For the purposes of this invention, spherical particles means solid or porous particles with a circularity parameter of at least 0.95. The circularity parameter is defined as the ratio of the circumference of a disk having the same area as the particle to the perimeter of the particle. A value of 1 characterizes particles that are perfectly spherical.

They preferably have an average size of less than 40 μm, preferably ranging from 1 to 20 μm, even more preferably from 2 to 10 μm.

Among the cellulose particles that can be used according to the invention, particular mention can be made of those marketed by Daito under the trademark CELLULOBEADS®, such as CELLULOBEADS USF® (D[50]=4 μm), CELLULOBEADS D-5® (D[50]<10 μm), CELLULOBEADS D-10® (D[50]<15 μm), CELLULOBEADS D-30® (D[50]<30 μm).

Preferably, the fillers are present in a content ranging from 0.1% to 20% by weight, relative to the total weight of the composition, preferably from 0.2% to 15% by weight, relative to the weight of the composition, preferably from 0.3% to 10% by weight, preferably from 0.4% to 5% by weight, and preferably from 0.5% to 4% by weight.

### Alpha Hydroxy Acid (AHA)

The composition according to the invention can comprise at least one AHA.

Alpha hydroxy acid refers according to the invention to a carboxylic acid having at least one hydroxy function occupying an alpha position on said acid (carbon adjacent to a carboxylic acid function). This acid may be presented in the final composition in the form of free acid and/or in the form of one of its associated salts (salts with an organic base or an alkali in particular), in particular according to the final pH imposed on the composition.

The α-hydroxy acids (alpha hydroxy acids or AHA) are, for example, chosen from lactic acid, citric acid, methyllactic acid, glucuronic acid, glycolic acid, pyruvic acid, 2-hydroxy-butanoic acid, 2-hydroxypentanoic acid, 2-hydroxyhexanoic acid, 2-hydroxyheptanoic acid, 2-hydroxyoctanoic acid, 2-hydroxy-nonanoic acid, 2-hydroxydecanoic acid, 2-hydroxyundecanoic acid, 2-hydroxydodecanoic acid, 2-hydroxytetradecanoic acid, 2-hydroxy-hexadecanoic acid, 2-hydroxyoctadecanoic acid, 2-hydroxytetra-cosanoic acid, 2-hydroxyeicosanoic acid; mandelic acid; phenyllactic acid; gluconic acid; galacturonic acid; aleuritic acid; ribonic acid; tartronic acid; tartaric acid; malic acid; fumaric acid; their salts and their mixtures.

According to a preferred embodiment, the alpha hydroxy acid is chosen from lactic acid, citric acid, malic acid, tartaric acids and their salts. More particularly, the alpha hydroxy acid is chosen from lactic acid, citric acid, their salts and mixtures thereof.

The one or more alpha hydroxy acids may be present in a content ranging from 0.001 to 10% by weight, from 0.005 to 5% by weight, and preferably from 0.01 to 3% by weight relative to the total weight of the composition.

### Physiologically Acceptable Aqueous Medium

The composition according to the invention comprises a physiologically acceptable aqueous medium. Said medium comprises water.

The water used can be sterile demineralized water and/or floral water such as rose water, cornflower water, chamomile water or linden water, and/or a spring or natural mineral water.

The composition preferably comprises at least 5% by weight of water, with respect to the total weight of the composition, preferably at least 10% by weight, preferably at least 20% by weight, preferably at least 30% by weight, and preferably at least 40% by weight. The composition preferably comprises from 5% to 95% by weight of water, with respect to the total weight of the composition, more preferably from 10% to 85%, even more preferably from 20% to 80%, even more preferably from 25% to 70%, even more preferably from 28% to 60%, and even more preferably from 30% to 50%.

The aqueous phase can also comprise at least one organic solvent miscible in water, at 25° C.

Preferably, the water-miscible organic solvent is chosen from alcohols, polyols and mixtures thereof.

Among the alcohols, mention can be made of C1-C10, more preferably C1-C5, alcohols, such as ethanol, isopropanol, propanol and butanol.

The polyol is, preferably, chosen from polyols having from 2 to 20 carbon atoms, preferably from 2 to 6 carbon atoms, such as glycerol, diglycerol, propyleneglycol, isoprene glycol, dipropyleneglycol, butylene glycol, hexylene glycol, 1,2-propanediol, 1,3-propanediol, pentylene glycol, polyethyleneglycols having from 2 to 200 ethylene oxide units and mixtures thereof.

The composition can comprise from 1% to 25% by weight of water-miscible organic solvent, with respect to the total weight of the composition, more preferably from 2% to 20% by weight, and even more preferably from 3% to 15% by weight.

### pH of the Composition

The composition according to the invention has a pH of less than or equal to 7, preferably of less than or equal to 6.5, and preferably of less than or equal to 6.3. Advantageously, the pH of the composition is between 3 and 6.3, preferably between 4 and 6.3.

Preferably, the cosmetic composition according to the invention comprises at least one base and/or at least one acid. The base and the acid according to the invention are known and conventionally used in the cosmetics field.

The base and/or the acid are in particular used to adjust the final pH of the composition to between 3 and 6.3.

The acid can, for example, be citric acid.

The base can be chosen from mineral bases such as for example alkali metal hydroxides, sodium hydroxide, potassium hydroxide.

Preferably, the base of the composition is an alkali metal hydroxide, preferably sodium hydroxide or potassium hydroxide.

The composition according to the invention can comprise at least one base at an active substance content ranging from 0.5% to 10% by weight, with respect to the total weight of the composition, particularly from 1% to 5% by weight, preferably ranging from 1% to 4% by weight.

The composition according to the invention can be obtained conventionally by those skilled in the art.

The following examples make it possible to understand the invention better, without being in any way limitative. The raw materials are referred to with the chemical or INCI name thereof. The amounts indicated are as a % by weight of raw materials with respect to the total weight of the composition (% w/w), unless specified otherwise.

### EXAMPLES: PREPARATION OF A COMPOSITION ACCORDING TO THE INVENTION AND COMPARISONS WITH COMPARATIVE COMPOSITIONS

Compositions B-J according to the invention, and comparative composition A, were prepared by mixing the ingredients in table 1.

The mixtures were heated to 60° C. during the mixing step to obtain homogeneity.

Protocol for Spreading the Compositions into a Film

The product is spread using a spreading bench (Elcometer 4340 Applicator) that allows the application speed and length to be adjusted. The bench is equipped with a suction system connected to a pump, so that the support on which the product is spread does not move.

Contrast charts with a black background and an unvarnished white background are used (1 byko-chart, uncoated N2A, code 2831). The spreading thickness can be adjusted using the square spreader placed on the support, so as to spread by leveling when the platform is switched on. Each section of the spreader allows the product to be spread to a different thickness, ranging from 25 μm to 200 μm. The chosen thickness is 25 μm to approximate the thickness of the film in vivo. A 960 g weight is added on top of the spreader during spreading. The spreading speed is set to 1 in/sec, i.e. 2.54 cm/s. The films are dried for 24 hours at ambient temperature and humidity (50% RH).

### Protocol for Testing Rub Resistance

The water rub resistance test is carried out by colorimetric measurements on a dry film before and after abrasion. Abrasion is carried out by attaching a strip of a cloth wipe (Chicopee Veraclean Polish Plus) that has been wet (with 400 μL of water in the cloth wipe) to the 25 μm spreader section. The 960 g weight is added on top of the spreader during abrasion. The bench speed is set at 2.54 cm/s.

The same spectrophotometric method is used to measure color before and after abrasion as explained above.

In order to assess rub resistance, the “Contrast Ratio”, respectively before rubbing (CR Dry Deposit, %) and after abrasion (CR Rub Dry Deposit, %) are measured. The loss [(CR Rub Dry Deposit−CR Dry Deposit]/CR Dry Deposit]*100, in percentage form, quantifies the loss of coverage and indicates the film's resistance to rubbing: the lower the loss, the more resistant the film is to rubbing.

Each CR value therefore represents an average of 3 to 6 measurements. The results are shown in Table 1.

TABLE 1

| | | B | C | D | E | F | G | H | I | J |
|---|---|---|---|---|---|---|---|---|---|---|
| | | according | according | according | according | according | according | according | according | according |
| Ingredients | A | to the | to the | to the | to the | to the | to the | to the | to the | to the |
| (% w/w) | comparative | invention | invention | invention | invention | invention | invention | invention | invention | invention |
| Chitosan | 2 | 2 | 2 | 2 | 2 | 2 | 2 | 2 | 2 | 2 |
| (Kiosmetine -CSH | | | | | | | | | | |
| by Kitozyme) | | | | | | | | | | |
| Lactic acid | 1 | 1 | 1 | 1 | 1 | 1 | 1 | 1 | 1 | 1 |
| Pigments* | 15 | 15 | 15 | 15 | 15 | 15 | 15 | 15 | 15 | 15 |
| Disodium | | 1 | | | | | | | | |
| stearoyl | | | | | | | | | | |
| glutamate | | | | | | | | | | |
| (AMISOFT | | | | | | | | | | |
| HS 21P) | | | | | | | | | | |
| Sodium | | | 1 | | | | | | | |
| lauroyl | | | | | | | | | | |
| glutamate | | | | | | | | | | |
| (AMISOFT | | | | | | | | | | |
| LS 11) | | | | | | | | | | |
| Sodium | | | | 1 | | | | | | |
| stearoyl | | | | | | | | | | |
| glutamate | | | | | | | | | | |
| (AMISOFT | | | | | | | | | | |
| HS 11 PF) | | | | | | | | | | |
| Oleic acid | | | | | 1 | | | | | |
| (EDENOR | | | | | | | | | | |
| OL 75 MY) | | | | | | | | | | |
| Isostearic | | | | | | 1 | | | | |
| acid | | | | | | | | | | |
| (PRISORINE | | | | | | | | | | |
| 3505-LQ- | | | | | | | | | | |
| (GD)) | | | | | | | | | | |
| Behenic acid | | | | | | | 1 | | | |
| (PALMERA | | | | | | | | | | |
| A8522) | | | | | | | | | | |
| Palmitic acid | | | | | | | | 1 | | |
| (EDENOR | | | | | | | | | | |
| C16-98 MY) | | | | | | | | | | |
| Glyceryl | | | | | | | | | 1 | |
| stearate | | | | | | | | | | |
| citrate | | | | | | | | | | |
| (AXOL C 62 | | | | | | | | | | |
| PELLETS MB) | | | | | | | | | | |
| Sodium | | | | | | | | | | 1 |
| coco-sulfate | | | | | | | | | | |
| (RHODAPON | | | | | | | | | | |
| CAS 100N MB) | | | | | | | | | | |
| Water | QS 100 | QS 100 | QS 100 | QS 100 | QS 100 | QS 100 | QS 100 | QS 100 | QS 100 | QS 100 |
| Friction loss | −54 (13) | −17(2) | −29 (8) | −23 (6) | −18 (4) | 2(1) | −23 (4) | −19 (4) | −21 (4) | −19(4) |
| with water | | | | | | | | | | |
| % (+/−%) | | | | | | | | | | |

*The pigments used in the compositions are a mixture of titanium dioxide, marketed under the trade name HOMBITAN FF PHARMA-VENATOR, red iron oxide, marketed under the trade name TAROX IRON OXIDE R-800HP - TITAN KOGYO, yellow iron oxide marketed under the trade name TAROX IRON OXIDE BL-100HPL- TITAN KOGYO and black iron oxide marketed under the trade name TAROX IRON OXIDE LL-100HP- TITAN KOGYO.

Results show better water resistance for compositions B, C, D, E, F, G, H, I and J according to the invention, compared with comparative composition A. The use of an anionic surfactant used in the invention thus provides water resistance.

Influence of the Anionic Surfactant Content on Water Resistance Compositions K, L, M and N according to the invention, and comparative composition O, were prepared by mixing the ingredients in table 2.

Compositions K, L, M and N according to the invention contain pigments and disodium stearoyl glutamate in a content ranging from 0.45% to 1.5% by weight relative to the total weight of the composition.

Comparative composition O contains chitosan in addition to pigments, but no anionic surfactants.

Water resistance is then assessed. The assessment protocol is as follows:
Apply each composition to an area of each forearm (with an additional forearm area as a control): to do this, 10 μl of each composition is applied to a forearm area measuring 2.5×2.5 cm for 15 seconds,
Leave to dry for 20 minutes,
Place 100 μL of water on a cotton rubbing cloth,
Apply the cotton cloth to the treated area using a 900 g weight for 5 seconds,
Remove the cotton cloth using a force of between 100 and 300 g, and
Visually assess the control deposit versus the water-rubbed deposit. This assessment includes a water resistance score, ranging from (−)=no water resistance, to (+++++)=excellent water resistance.

TABLE 2

| | | K | L | M | N |
|---|---|---|---|---|---|
| | O | according | according | according | according |
| Ingredients | compar- | to the | to the | to the | to the |
| (% w/w) | ative | invention | invention | invention | invention |
| Chitosan | 2 | 2 | 2 | 2 | 2 |
| (Kiosmetine- | | | | | |
| CSH by | | | | | |
| Kitozyme) | | | | | |
| Lactic acid | 1 | 1 | 1 | 1 | 1 |
| Pigments* | 15 | 15 | 15 | 15 | 15 |
| Disodium | | 0.45 | 0.75 | 1 | 1.5 |
| stearoyl | | | | | |
| glutamate | | | | | |
| (AMISOFT HS | | | | | |
| 21P) | | | | | |
| Water | QS 100 | QS 100 | QS 100 | QS 100 | QS 100 |
| Water | 1 | 3 | 4 | 4 | 5 |
| resistance | | | | | |
| score (from 1 | | | | | |
| (−) to 5 (+)) | | | | | |

*The pigments used in the compositions are a mixture of titanium dioxide, marketed under the trade name HOMBITAN FF PHARMA-VENATOR, red iron oxide, marketed under the trade name TAROX IRON OXIDE R-800HP - TITAN KOGYO, yellow iron oxide marketed under the trade name TAROX IRON OXIDE BL-100HPL- TITAN KOGYO and black iron oxide marketed under the trade name TAROX IRON OXIDE LL-100HP- TITAN KOGYO.

The results show improved water resistance for compositions K, L, M and N according to the invention, compared with the comparative composition O. Improved water resistance is observed when the amount of anionic surfactant is 1.5%. Use of a higher amount (1.5%) of anionic surfactant in the composition according to the invention thus provides improved water resistance.

### Influence of the Pigment Content on Water Resistance and Coverage Properties

Compositions R, S and T according to the invention, and comparative compositions P and Q were prepared by mixing the ingredients of table 3.

Compositions R, S and T according to the invention comprise pigments in a content ranging from 1% to 70% % by weight relative to the total weight of the composition.

Comparative composition P comprises less than 1% by weight relative to the total weight of the composition of pigments (i.e. 0.5%).

Comparative composition Q comprises more than 70% by weight relative to the total weight of the composition of pigments (i.e. 72%).

The water rub resistance and coverage properties are assessed by colorimetric measurements on a dry film before and after abrasion following the same protocol as detailed above.

TABLE 3

| | | | R | | S | | T |
|---|---|---|---|---|---|---|---|
| | P | Q | according | | according | | according |
| Ingredients | compar- | compar- | to the | | to the | | to the |
| (% w/w) | ative | ative | invention | | invention | | invention |
| Chitosan | 2 | 0.48 | 2 | | 2 | | 0.48 |
| (Kiosmetine- | | | | | | | |
| CSH by | | | | | | | |
| Kitozyme) | | | | | | | |
| Lactic acid | 1 | 0.24 | 1 | | 1 | | 0.24 |
| Pigments* | 0.5 | 72 | 2 | | 15 | | 62 |
| Disodium | 0.45 | 0.25 | 0.45 | | 1 | | 0.25 |
| stearoyl | | | | | | | |
| glutamate | | | | | | | |
| (AMISOFT | | | | | | | |
| HS 21P) | | | | | | | |
| Water | Qs 100 | Qs 100 | Qs 100 | | Qs 100 | | Qs 100 |
| Friction loss | — | — | −17 | (3) | −17 | (2) | −3 (1) |
| with water % | | | | | | | |
| (+/− %) | | | | | | | |
| Coverage | 5 (1) | — | 24 | (4) | 73 | (5) | 99 (1) |
| CR % (+/− %) | | | | | | | |

*The pigments used in the compositions are a mixture of titanium dioxide, marketed under the trade name HOMBITAN FF PHARMA-VENATOR, red iron oxide, marketed under the trade name TAROX IRON OXIDE R-800HP - TITAN KOGYO, yellow iron oxide marketed under the trade name TAROX IRON OXIDE BL-100HPL- TITAN KOGYO and black iron oxide marketed under the trade name TAROX IRON OXIDE LL-100HP- TITAN KOGYO.

Compositions R, S and T according to the invention show a high level of coverage and good water resistance, significantly better than comparative compositions A, P or Q.

The results show that it is not possible to formulate compositions with more than 70% by weight of pigments. Indeed, comparative composition Q is solid and heterogeneous, and not formulable.

Comparative composition P shows that it is necessary to introduce at least 1% by weight of pigments to achieve a minimum coverage. Indeed, the initial CR is too low.

1. A cosmetic composition comprising, in a physiologically acceptable aqueous medium:
a) at least 0.01% by weight of native chitosan, with respect to the total weight of the composition, which native chitosan has a molecular weight strictly greater than 3000 Daltons, this amount being strictly less than 15% by weight, and
b) at least one compound chosen from among anionic surfactants of the carboxylate, phosphate, sulfonate, or sulfate type and/or mixtures thereof.

2. The composition according to claim 1, wherein the native chitosan has a molecular weight greater than or equal to 10 kDa.

3. The composition according to claim 1, wherein the degree of acetylation of the native chitosan is less than or equal to 80%.

4. The composition according to claim 1, wherein the native chitosan is a polysaccharide prepared from a fungal source.

5. The composition according to claim 1, wherein the native chitosan is present in an amount ranging from 0.01% to 14% by weight.

6. The composition according to claim 1, comprising at least one compound chosen from pigmentary coloring matters and fillers.

7. The composition according to claim 6, wherein the pigmentary coloring matter is a mineral pigment chosen from titanium dioxide, iron oxides, zirconium or cerium oxides, zinc or chromium oxides, manganese violet, ultramarine blue, chromium hydrate and iron blue, metallic powders; and/or the filler is chosen from cellulose particles, silicas, starch, kaolin, clays, nylon and mixtures thereof.

8. The composition according to claim 6, wherein the pigmentary coloring matter is an organic pigment chosen from the compounds nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, and quinophthalone.

9. The composition according to claim 6, wherein the pigmentary coloring matter is chosen from mineral pigments.

10. The composition according to claim 6, wherein the pigmentary coloring matter is present in an amount ranging from 1% to 70% by weight.

11. The composition according to claim 1, wherein the compound chosen from the anionic surfactants is a carboxylate anionic surfactant chosen from citrates, fatty acids, N-acyl amino acids, lactylates and/or their salts.

12. The composition according to claim 1, wherein the carboxylate anionic surfactant is chosen from fatty acids and/or their salts.

13. The composition according to claim 1, wherein the anionic surfactant is an N-acyl amino acid comprising an acyl group having from 8 to 22 carbon atoms.

14. The composition according to claim 1, wherein the anionic surfactant is disodium stearoyl glutamate.

15. The composition according to claim 1, wherein the anionic surfactant is chosen from alkyl sulfosuccinates, alkyl ether sulfosuccinates, and alkyl amide sulfosuccinates.

16. The composition according to claim 1, wherein the anionic surfactant chosen from citrates, fatty acids, N-acyl amino acids, lactylates and/or salts thereof is present in the composition in a content ranging from 0.1% to 10% by weight relative to the total weight of the composition.

17. The composition according to claim 1, wherein the physiologically acceptable aqueous medium comprises at least 5% by weight of water, with respect to the total weight of the composition; and optionally at least one organic solvent miscible in water, at 25° C., chosen from alcohols, polyols and mixtures thereof.

18. The composition according to claim 1, wherein the weight ratio of chitosan to anionic surfactant (i.e. the weight ratio chitosan:anionic surfactant) is between 0.1 and 10.

19. The composition according to claim 1, comprising at least one alpha hydroxy acid; their salts and their mixtures.

20. The composition according to claim 1 which is substantially free from (meth)acrylic polymer and/or silicone.

21. The composition according to claim 1, which has a pH less than or equal to 7.

22. A method for making up and/or caring for the skin and/or keratinous appendages, wherein the composition according to claim 1 is applied to the skin and/or keratinous appendages.
